(19) United States
(12) Patent Application Publication
(10) Pub. No.: US 2004/0202571 A1
(43) Pub. Date: Oct. 14, 2004

(54) Directed medication system and method

(76) Inventor: Gary R. Epler, Wellesley, MA (US)

Correspondence Address: MESMER & DELEAULT, PLLC, 41 BROOK STREET, MANCHESTER, NH, 03104, US

(21) Appl. No.: 10/616,139

(22) Filed: Jul. 9, 2003

Related U.S. Application Data
(63) Continuation-in-part of application No. 10/411,459, filed on Apr. 10, 2003.

(51) Int. Cl. G01N 33/48; A61B 10/00

(52) U.S. Cl. 422/61; 436/63; 600/572

(57) ABSTRACT

A directed medication system and method having a drug metabolism test component and a prescription instruction component. The drug metabolism test component includes an apparatus to receive a user's biological sample for testing to determine the presence of one or more predefined drug metabolism markers. The prescription instruction component includes at least coordinating instructions disclosing to a user of the drug metabolism test component the procedure for obtaining a customized medical therapy based on the results of the drug metabolism test component. The prescription instruction component preferably includes a prescription for a customized medical therapy based on the results of the drug metabolism test.

This application is a continuation-in-part of application Ser. No. 10/411,459, filed Apr. 10, 2003.

### BACKGROUND OF THE INVENTION

1. Field of the Invention

The present invention relates generally to medical diagnostic test kits. Particularly, the present invention relates to a system to minimize adverse drug events and to maximize drug effectiveness.

2. Description of the Prior Art

Numerous medical tests are commercially available for use in the home. Two illustrative examples include medical test kits that are used to discover an individual's blood sugar, i.e. glucose, level at a particular time, or an individual's hormone level at a particular time for pregnancy testing.

The glucose test kit allows a diabetic to test his/her blood sugar and adjust his/her daily insulin dosages accordingly without consulting a doctor or other medical personnel. To initially learn to take blood and use a personal glucose test kit, a diabetic may get trained by a nurse or other medical personnel. Not all personal test kits require the taking of blood or other internal body fluid. For example, personal pregnancy test kits are commercially available that allows a woman, in the privacy of her own home, to test her urine for the presence of a pregnancy hormone, human chorionic gonadotropin.

DNA test kits that are used to determine familial relationships are also commercially available. DNA test kits use DNA sequencing technology to determine the familial relationship of an individual. DNA sequencing is the determination of the order of nucleotides (the base sequence) that exists in a DNA molecule of an individual.

As one example of DNA lineage testing, GeneTree™ offers DNA Personal Paternity Tests. DNA Personal Paternity Tests can be used in the home to statistically determine the likelihood of a particular man being a child's father. The GeneTree™ DNA Personal Paternity Tests utilize cheek cells collected with colored swabs where one color indicates an alleged father and a second color indicates the subject child. The colored swabs are placed in coordinating colored envelopes that match the color of the user's swab. The envelopes are then sent to a laboratory where genetic testing is performed according to standard procedures.

Unfortunately, most medical test kits on the market today are used for determining events that have already occurred. These test kits determine the existence of a condition such as low blood sugar, pregnancy, etc., or to determine the statistical probability of a genetic link between two or more individuals.

There exists today a problem with the system used for the development of new drugs and drug treatments. Currently, pharmaceutical companies are limited to developing drugs using a one-size-fits-all system. This system allows for the development of drugs to which the average patient will respond. Unfortunately, some patients have a severe negative reaction to a prescribed drug while some patients may have no response to the medication at all. In the industry, this is called an adverse drug reaction.

A 1998 study of hospitalized patients published in the Journal of the American Medical Association reported that in 1994, adverse drug reactions accounted for more than 2.2 million serious cases and over 100,000 deaths. An adverse drug reaction is one of the leading causes of hospitalization and death in the United States. Currently, there is no simple way to determine if people will respond well, badly or not at all to a medication.

With the advent of the Internet and the proliferation of medical-related information, patients are becoming more aware of the seriousness of adverse drug reactions. They may even know someone who has suffered such an event. Harvard Business School Professor Regina Herzlinger, writing in the July 2002 issue of the Harvard Business Review, reports that patients are demanding better, more tailored treatments. The article further notes that a report in the Journal of the American Medical Association showed that screening drugs against a person's genetic makeup could reduce many dangerous reactions. The AMA report revealed that more than half of the 27 drugs frequently cited for causing adverse reactions were linked to genetic variations in a patient's ability to metabolize the drugs.

Drug metabolism in the body makes drugs more readily excreted in the urine or bile. Many drugs are metabolized in the liver or kidney and the function of these organs along with certain enzymes can affect drug metabolism. One common way of metabolizing drugs involves the cytochrome P450 enzymes. Many drug interactions are a result of inhibition or induction of cytochrome P450 enzymes that increase or decrease the retention of drugs in the body. A physician can better anticipate and manage adverse drug reactions in a patient by knowing an individuals genetic variation regarding the cytochrome P450 enzymes.

Therefore what is needed is a directed medication system and method that is minimally invasive. What is also needed is a directed medication system and method that is used to predict a patient's potential of an adverse medical reaction to a particular drug treatment/medical therapy. What is even further needed is a directed medication system and method that is used to predict the efficacy of a particular medication/medical therapy within the user's body. What is still further needed is a directed medication system and method that minimizes the potential for an adverse drug event.

### SUMMARY OF THE INVENTION

It is an object of the present invention to provide a directed medication system and method. It is also an object of the present invention to provide a directed medication system and method that can be utilized by any individual. It is a further object of the present invention to provide a directed medication system and method that is minimally invasive. It is still a further object of the present invention to provide a directed medication system and method that will predict the likelihood of a patient's adverse medical reaction to a particular drug treatment/medical therapy. It is another object of the present invention to provide a directed medication system and method that will predict the efficacy of medications within the user's body. It is yet another object of the present invention to provide a directed medication system and method for determining the likelihood of potential liver dysfunction in individuals as well as cardiac toxicity, pulmonary toxicity and other organ toxicity.

The present invention achieves these and other objectives by providing a directed medication system and method that includes a drug metabolism test component and a prescription instruction component. The drug metabolism test component includes a medical test kit for determining the function of internal organs, or the presence of particular enzymes or blood and urine chemistry markers associated with drug metabolism.

The preferred drug metabolism test component includes a sample container comprising a hinged folder having a first and a second sample holding pads, a first and a second sample collection devices corresponding to the first and second sample holding pads, and an instruction sheet. The test component may also include a desiccant pillow and an outer pouch sized to contain both the utilized sample container and desiccant pillow.

The hinged folder of the sample container has a first surface with locations for the first and second sample holding pads, and a second surface that overlays the first surface. The sample holding pads of the sample container first surface are protected by the second surface. The sample container first surface has indicia markings below the first and second sample holding pads. The indicia markings indicate the proper location for placing a first and a second cell sample upon the appropriate sample holding pad. The first and second surfaces of the sample container are approximately of equal size.

The second surface may have an extended portion, i.e. a securing tab, for securing the second surface to the first surface. The securing tab may either fold over a lower portion end of the first surface or may be inserted into a slit of the first surface below the first and second sample holding pads. The securing tab may also have a pressure-sensitive adhesive to secure the tab against the back side of the first surface of the sample container near the lower portion end of the first sample container surface below the first and second sample holding pads. Alternatively, the second surface may have a coating of a pressure-sensitive adhesive along a portion adjacent an edge of the second surface for adhering to the first surface.

The sample holding pads on the first surface of the sample container are sized and shaped to receive enough quantity of biological sample to achieve accurate results for the desired test. In the preferred embodiment, the biological sample is a buccal cell sample. In this instance, it is to determine the presence of risk markers in a person's DNA that predicts a high probability of possible liver dysfunction or other organ dysfunction leading to an adverse drug reaction. First and second sample holding pads may be any shape and may each be a different shape. The sample holding pads are absorbent and impregnated with chemicals to lyse cell membranes on contact, to immobilize and stabilize nucleic acids (DNA and RNA), and to inactivate bacteria and viruses. The sample holding pads allow biological cell samples to be collected, transported and stored at room temperature.

The first and second sample collection devices are cell collection devices and specifically correspond to the first and second sample holding pads. The first and second cell collection devices are generally flat, elongated rectangular swabs composed of an inert, i.e. non-reactive, material that will not introduce contaminants onto the sample holding pad. The cell collection devices have indicia markings on both sides that indicate the correct location of finger placement to pick up and utilize the device, and the location of cell collection. The indicia for locating finger placement are important to prevent inadvertent sample contamination to the first and second cell collection locations on the cell collection device. Examples of contaminants that can damage the cell collection location include finger oils and dirt, hand soap and hand lotion residues, fingernail polishes and other cosmetic product residues commonly found on the fingers or on the hand of a home user. The cell collection locations of the cell collection devices may also contain outer protective wrappings to protect the cell collection locations from external contamination until the user understands the instructions and is ready to proceed with cell collection.

The instruction sheet of the test component may be either a separate sheet within the component or may be an integral part of one of the surfaces of the sample container. For instance, the instructions may be printed on the outside surface of the foldable sample container. The instructions provide a sequence of detailed informative steps teaching the proper use of the test component to achieve accurate results. For example, detailed information about the proper method of picking up the first cell collection device, obtaining a sample from the inside surface of a user's first cheek, placing the cell sample onto the indicia coordinated first holding pad of the sample container, and repeating the procedure for the same first cheek using the reverse side of the first collection device. Next, the previously described procedure is then followed for obtaining a sample for the inside surface of the user's second cheek using the second cell collection device and transferring the sample to the second holding pad of the sample container. Should a user be left-handed instead of right-handed, additional instruction about changes to the method of picking up and using a first and second cell collection device for use by a left-handed user are given. It is understood that obtaining a DNA sample is not limited to a buccal cell sample but can include DNA obtained from, but not limited to, other sources such as blood, urine, hair, skin, and other body fluids. If the cell collection locations on the cell collection devices contain outer protective wrappings, the instruction sheet would also contain additional instructions about the proper time of removal and method of removal of the outer protective wrapping before cell collection. The additional instructions about the outer protective wrapping would be inserted at the appropriate location within the sequence of instructions.

An outer pouch of the test component is sized to contain the sample container, which has coded indicia markings that indicate the identity of the user of the test component. The coded indicia markings may be used for several purposes such as, for example, maintaining user privacy, maintaining correct sample identity during preliminary sample preparation and analysis by the testing laboratory, and returning the specific genomic test results to the correct user.

A desiccant pillow of the test component may be included to maintain a relatively dry atmosphere within the outer pouch. The desiccant pillow includes a material for absorbing excess moisture from the atmosphere in the pouch. This prevents moisture from adversely affecting the cell samples that are disposed onto the sample holding pads located inside the folded sample container.

The drug metabolism test component incorporates indicia to alert a user that the test component is used to identify risk markers for predicting the probability of an adverse drug reaction. Particularly, it is the risk markers associated with the cytochrome P450 enzyme family. The cytochrome P450 enzymes are used in the metabolic processing of medication. Cytochrome P450 enzymes include the CYP3A gene family and CYP2D6. Identification of a user's risk markers is important because adverse drug reactions can result in liver dysfunction in some individuals. The risk markers are identified by examination of an individual's structural genes. In addition to the examination of the structural gene, genotypic testing of the regulatory/promoter regions, certain transcription factors and the gene sequences important for correct splicing will be performed. By this examination, the risk of dangerously large changes in liver function will be determined. By knowing what risk markers are present, the genomic analysis can be correlated to an effective drug therapy that minimizes the potential for an adverse drug reaction.

In order to maintain user privacy, a peelable label containing tracking indicia is removably affixed to the back side of the sample container. The sample container also includes matching tracking indicia to that on the peelable label. Each test component has a unique tracking indicia.

The prescription instruction component directs the user/patient on how to obtain an initial dose of medical therapy for a particular affliction and a customized medical therapy based on the test results of the drug metabolism test component. The prescription instruction component can be provided with the drug metabolism test component as a companion product or located separate from the test component at a healthcare provider's office such as a doctor. The healthcare provider can dispense the test component with the prescription instruction component. The healthcare provider can dispense the prescription instruction component instructions separate from the test component. If the test component is not given to the user/patient by the healthcare provider, the prescription instruction component may contain instructions or directions that provide the user/patient with informative steps on how to obtain the drug metabolism test component over-the-counter or from a pharmacy by prescription. The prescription instruction component also explains how to obtain the initial dose of medical therapy, and a customized dose of medical therapy based on the results of the drug metabolism test component.

For example, the instructions may direct the user/patient to take the initial dose of medication if an initial dose is provided when receiving the drug metabolism test component or separately dispensed by a doctor. The prescription instruction component may direct the user/patient to obtain the initial dose by a prescription issued by a healthcare provider concomitantly with the drug metabolism test component, or to have a healthcare provider communicate with other healthcare providers as necessary to obtain the initial dose from a pharmacy. The communication between or among healthcare providers can include a prescription by telephone, facsimile, computer or any other means used to communicate a prescription. This communication can be transferred by telephone, facsimile, computer or any other means used to communicate a prescription.

Also, the prescription instruction component directs the patient/user to only obtain, and healthcare providers to only dispense the customized dose of medical therapy based on the results of the drug metabolism test after the test results have been communicated to the appropriate healthcare provider. The healthcare provider may be, but is not limited to, a physician, dentist, nurse, pharmacist, physician's assistant, or nurse practitioner.

In one embodiment, the directed medication system and method incorporates an initial dose of medical therapy and a prescription for a customized medical therapy. In a second embodiment, the directed medication system and method incorporates only a combined prescription for an initial dose of medical therapy and for a customized medical therapy. In a third embodiment, the directed medication system and method incorporates separate prescriptions for an initial dose of medical therapy and for a customized medical therapy. In a fourth embodiment, the directed medication system and method incorporates only instructions for obtaining an initial dose of medical therapy and a customized medical therapy. In yet another embodiment, the directed medication system and method incorporates an initial dose of medical therapy and instructions for obtaining a customized medical therapy.

The prescription for an initial dose of medication may be separate from or combined with a prescription for a customized medical therapy that is based on the results of the drug metabolism analysis. The prescription for a customized medical therapy may be separate from or combined with a prescription for an initial dose of medical therapy. The prescriptions for an initial dose of medication and for a customized medical therapy may be written or communicated by telephone, facsimile, computer or any other means used to communicate a prescription.

The prescription for customized medical therapy may be an individual prescription having multiple medical therapy dosage schedule parameters where each dosage schedule parameter is related to the results of the drug metabolism analysis. In the alternative, the prescription for customized medical therapy may involve separate prescriptions where each prescription has an individual medical therapy dosage schedule parameter related to the results of the drug metabolism analysis. The customized medical therapy dosage schedule parameters include a homozygous positive pattern that indicates dispensing of the prescribed dose, a heterozygous mid-positive pattern that indicates dispensing of an adjusted dose, and a negative pattern that indicates dispensing of a further adjusted dose or that a new medication should be dispensed. The customized medical therapy dosing schedule parameters may include indicia that are letters, words, symbols, colors or any combination thereof.

### BRIEF DESCRIPTION OF THE DRAWINGS

FIG. 1 is a perspective view of one embodiment of the directed medication system of the present invention.

FIG. 2 is a top plan view of one embodiment of the test component of the present invention.

FIGS. 3 and 4 are top plan views of the embodiment in FIG. 2 showing different shaped sample holding pads.

FIG. 5 is a back plan view of the embodiment in FIG. 2 showing the tracking indicia.

FIGS. 6A and 6B are enlarged front plan views of the embodiment in FIG. 2 showing the indicia on the buccal cell swabs.

FIGS. 7 is a top plan view of one embodiment of the prescription instruction component of the present invention.

FIG. 8 is a top plan view of another embodiment of the prescription instruction component of the present invention showing a prescription for a customized medical therapy.

FIGS. 9A and 9B are top plan views of another embodiment of the prescription in FIG. 8 showing multiple dosage schedule parameters for a customized medical therapy.

FIGS. 10 is a top plan view of the initial dose prescription of the present invention for a customized medical therapy.

FIG. 11 is a top plan view of the prescription instruction component of the present invention showing an initial dose and a conditioned prescription having multiple dosing schedule parameters for a customized medical therapy based on the results of the drug metabolism test component.

### DETAILED DESCRIPTION OF THE PREFERRED EMBODIMENT

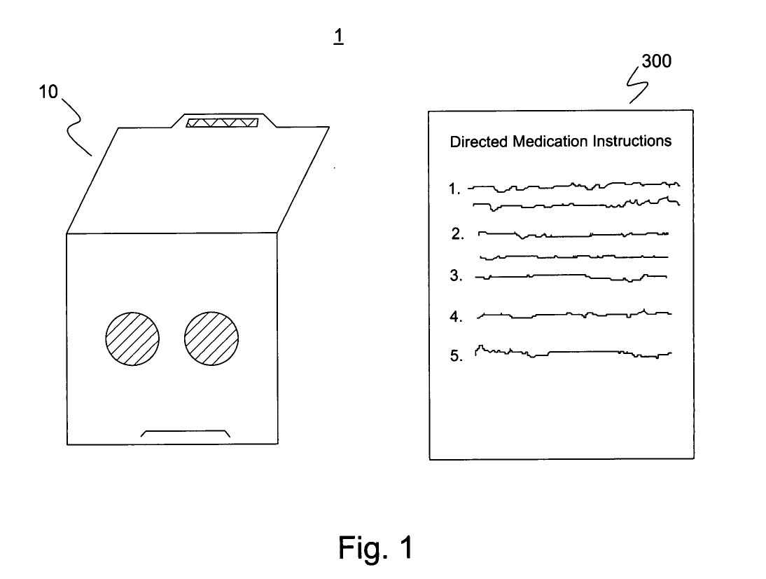

The preferred embodiment(s) of the present invention are illustrated in FIGS. 1-11. FIG. 1 illustrates the broadest conceptualization of the directed medication system 1. System 1 includes a drug metabolism test component 10 and a prescription instruction component 300. Drug metabolism test component 10 is used to receive a patient/user biological sample that is analyzed providing a drug metabolism test result. Preferably, drug metabolism test component 10 is a genomics-based test. Prescription instruction component 300 has a fulfillment characteristic that is directed by the results obtained from the genomics-based test component 300.

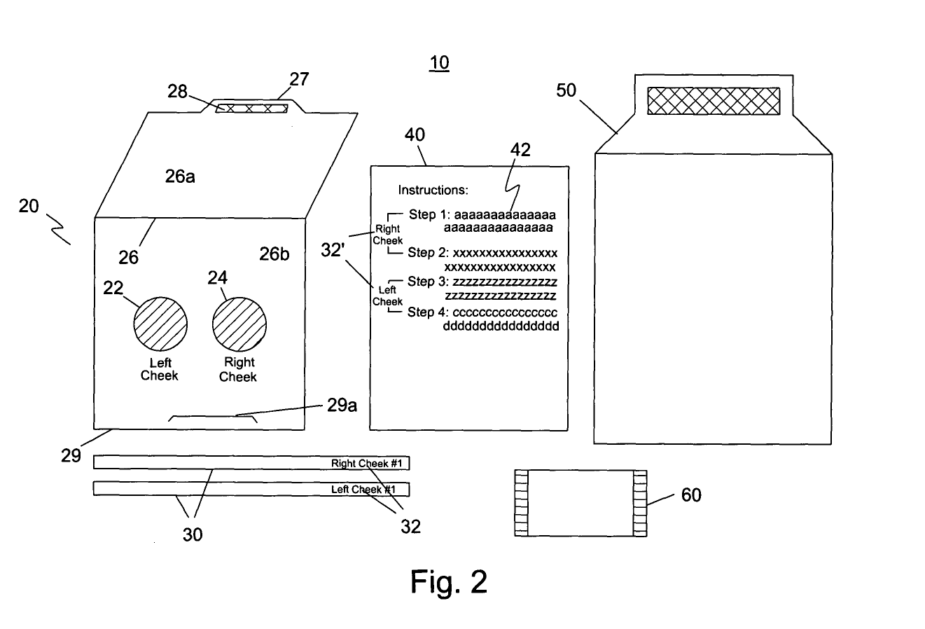

FIG. 2 illustrates front planar views of the preferred genomics-based test component 10. The genomics test component 10 is specific for determining the presence of adverse drug reaction risk markers in a user's DNA. Genomics-based test component 10 includes a sample container 20, a pair of buccal cell swabs 30, instruction sheet 40, an outer pouch 50, and a desiccant 60. Sample container 20 includes a first sample holding pad 22 and a second sample holding pad 24. Sample container 20 is generally a hinged folder having a creased fold 26. Creased fold 26 divides sample container 20 into an upper portion 26a and a lower portion 26b of approximately equal size. Upper portion 26a has a securing tab 27 that may either fold over lower portion end 29 of lower portion 26b or inserted into a lower portion slit 29a. Securing tab 27 may also have a pressure-sensitive adhesive 28 to secure tab 27 against the back side (not shown) of lower portion 26b near lower portion end 29.

Sample holding pads 22 and 24 are generally impregnated with chemicals to lyse cell membranes and immobilize nucleic acids. Buccal cell swabs 30 are used for obtaining a buccal cell sample. Typically, buccal cell swabs 30 are made of wood to scrape a sample of epithelial cells from the inside cheek of a user. It is particularly important to include indicia 32 on each of cell swabs 30 to prevent sample contamination and to increase the probability of proper user compliance. Swabs 30 include indicia 32 on each side of the swab. Indicia 32 link the test sample to be collected with the associated sample holding pad 22 or 24.

Test component instruction sheet 40 includes instructions 42 that provide a sequence of detailed informative steps instructing in the proper use of the test component 10 to achieve accurate adverse drug reaction risk marker test results. The instructions include the use of linking indicia 32′ to guide the user in use of swabs 30 and proper sample collection and transfer techniques as well as disclosing the type of test component, i.e. to identify in a person's DNA the presence of risk markers that increase the probability of a particular drug causing liver dysfunction and leading to an adverse drug reaction. Test component instructions 42 provide detailed information about picking up the first swab or cell collection device 30, obtaining a sample from the inside surface of a user's first cheek and placing the obtained sample onto the proper holding pad 22, 24 as identified by the indicia 32 and 32′.

Outer pouch 50 is a sealable container sized to receive sample container for transfer to another location. Desiccant 60 is a standard moisture-absorbent pillow sized for placement within outer pouch 50 along with sample container 20. Desiccant 60, when placed within outer pouch 50 with sample container 20, maintains a relatively dry atmosphere within outer pouch 50. The desiccant material is typically a substance having a high affinity to water molecules that binds and holds the water molecules found in the atmosphere within outer pouch 50.

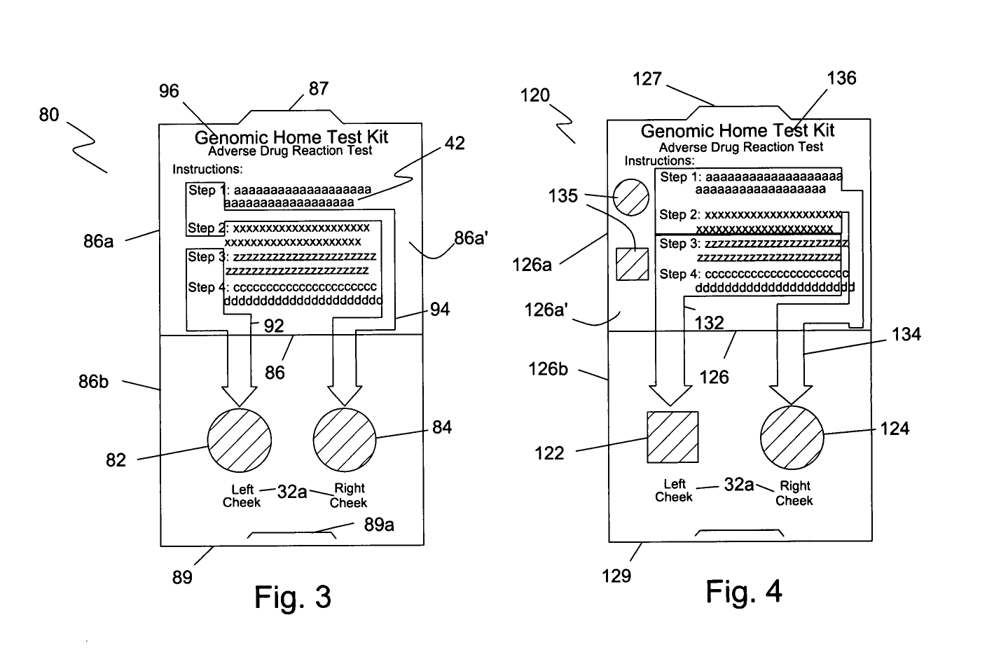

Turning now to FIG. 3, there is shown another embodiment of the sample container of the present invention. Sample container 80 includes a first sample holding pad 82 and a second sample holding pad 84. Sample container 80 is generally a hinged folder having a creased fold 86. Creased fold 86 divides sample container 80 into an upper portion 86a and a lower portion 86b of approximately equal size. Upper portion 86a has a securing tab 87 that may either fold over lower portion end 89 of lower portion 86b or inserted into a lower portion slit 89a.

In this embodiment, test component instructions 42 are imprinted on a second surface 86a′ of upper portion 86a. Indicating indicia 92 and 94 links the sampling instructions 42 for first and second cheek samples with the properly identified first and second holding pads 82, 84. Sample container 80 also has indicia 96 clearly identifying the adverse drug reaction risk marker kit.

FIG. 4 shows yet another embodiment of the sample container of the present invention. Sample container 120 includes a first sample holding pad 122 and a second sample holding pad 124. Sample container 120 is generally a hinged folder having a creased fold 126. Creased fold 126 divides sample container 120 into an upper portion 126a and a lower portion 126b of approximately equal size. Upper portion 126a has a securing tab 127 that may either fold over lower portion end 129 of lower portion 126b or insert into a lower portion slit 129a.

Like the embodiment in FIG. 3, test component instructions 42 are imprinted on a second surface 126a′ of upper portion 126a. Indicating indicia 132 and 134 may also connect the sampling instructions 42 for right and left cheek samples with the properly identified first and second holding pads 122,124. Additional linking indicia 135 may be used to further link relevant portions of instructions 42 to sample holding pads 122 and 124. Sample container 120 also has indicia 136 clearly identifying the adverse drug reaction risk marker kit. Sample container 120 further includes a first holding pad 122 that has a different shape than second holding pad 124. In this example, the first holding pad 122, labeled Left Cheek, has a square shape. Second holding pad 124, labeled Right Cheek, has a circular shape. This shape differentiating indicia 135 is also included on swabs 30, which further helps the user in reducing contamination by visually connecting the additional indicating indicia to the swabs 30 for use with the proper holding pad 122 or 124.

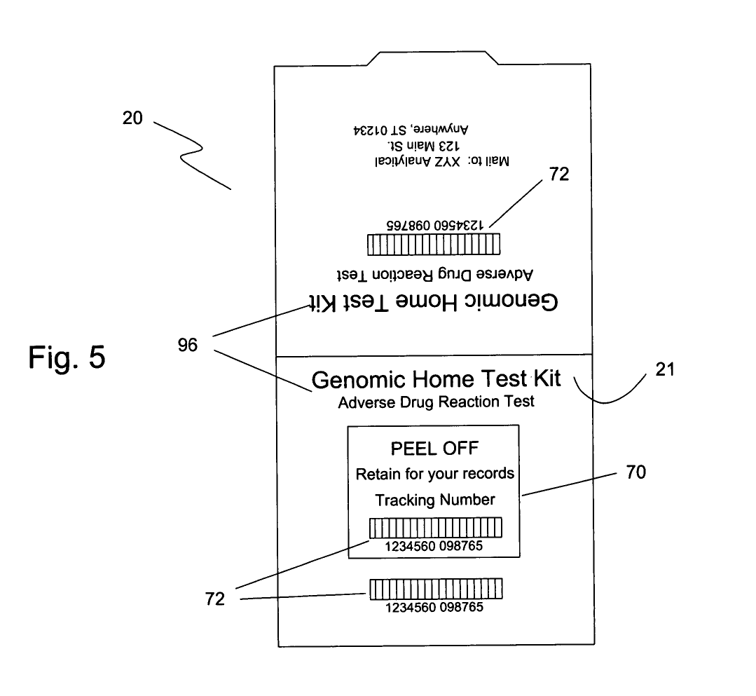

FIG. 5 is a back view of sample container 20. On a back surface 21, there is imprinted indicia 96 to indicate the type of test, indicia 72 for tracking the sample, a mailing address of the place for analysis, and a removable, peel-off sticker 70. Removable sticker 70 includes tracking indicia 72 for the user to obtain the results of the test while maintaining user privacy.

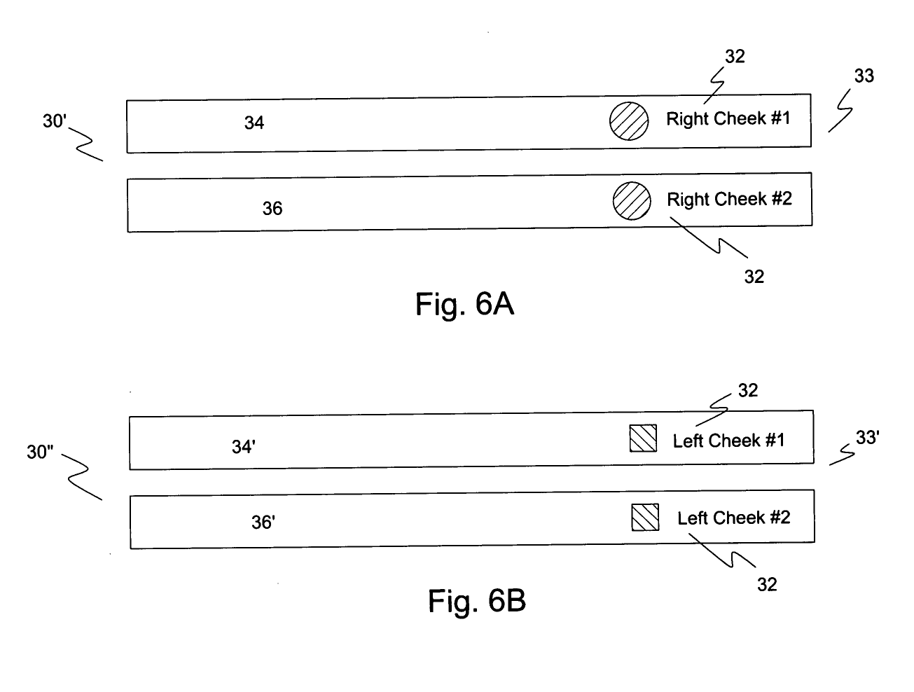

FIGS. 6A and 6B illustrate the indicating indicia on swabs 30. Turning now to FIG. 6A, right cheek swab 30′ has a first swab side 34 and a second swab side 36. Swab sides 34, 36 have imprinted thereon adjacent a swab end 33, indicia 32 indicating the sample holding pad associated with right cheek swab 30′. In this particular example both the shape and the word symbols indicate the proper sample holding pad for receiving the right cheek sample. In FIG. 6B, left cheek swab 30″ also has a first swab side 34′ and a second swab side 36′. Adjacent a swab end 33′, indicia 32 are imprinted to indicate the sample holding pad associated with left cheek swab 30″.

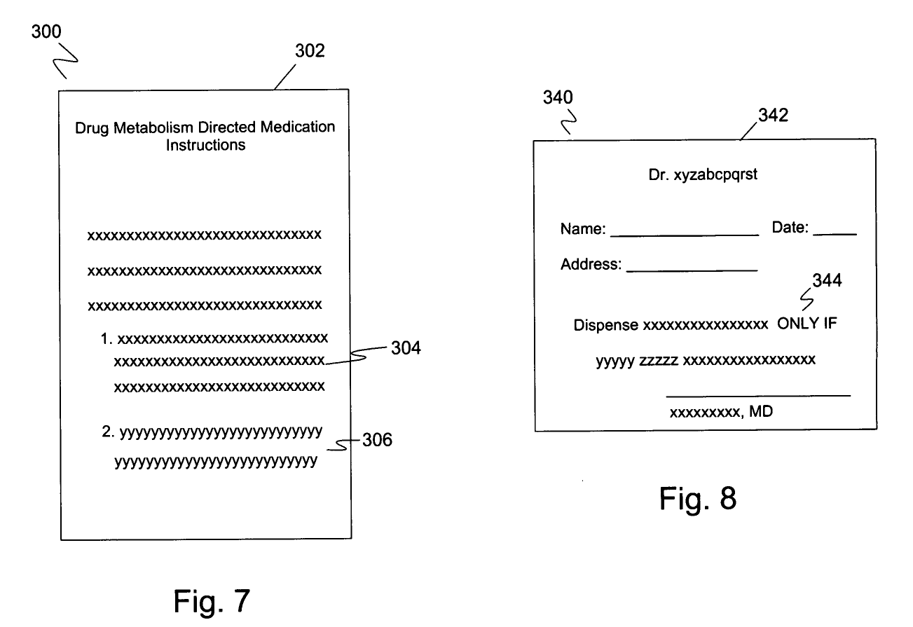

Turning now to FIGS. 7-11, there is illustrated various embodiments of the prescription instruction component 300 of the directed medication system I that includes an instruction component 302, a prescription component 340, or both. Instruction component 302 may be verbal instructions provided by the healthcare provider to the patient, but is preferably written instructions provided to the patient. FIG. 7 illustrates one embodiment of a basic prescription instruction component 300 comprising only of an instruction component 302 that is received by a patient from a healthcare provider. In this embodiment, prescription instruction component 300 includes a first instruction 304 that directs the patient to obtain a drug metabolism test component 10 and to follow the test component instructions to submit a test sample for testing. A second instruction 306 directs the user/patient to obtain a customized medical therapy after the results of test component 10 are presented to the user/patient's healthcare provider.

The manner in which the results of test component 10 are presented to the healthcare provider may vary. For example, the test results may be sent directly to the patient. In this case, the patient would present the test results to an appropriate healthcare provider to obtain the proper medical therapy. The test results may also be communicated directly to the patient's healthcare provider. This would likely improve efficiency of the directed medication process. Another alternative would be to have the test results sent directly to the pharmacy. Depending on the particular embodiment of the directed medication system, the pharmacist would either fill an existing, conditional prescription based on the results of the test or call the patient's healthcare provider and communicate the test results to the healthcare provider at which time the healthcare provider would issue a customized medical therapy to the pharmacist based on the test results. The pharmacist would then dispense the customized medical therapy to the patient.

Instruction component 302 may be modified in accordance with the embodiment used to carry out the method of the present invention. For instance, the drug metabolism test component 10 may be an over-the-counter item or may be obtained by prescription or directly from a healthcare provider. Further, the prescription component 340 may be included in various embodiments. All of these variations will impact the structure/arrangement of the prescription instruction component 300.

FIG. 8 illustrates an embodiment of the prescription instruction component 300 that includes a prescription component 340. In this embodiment, prescription component 340 is a prescription 342 issued by a healthcare provider. Prescription 342 includes a conditional prescription portion 344 instructing the pharmacist to dispense the medical therapy pending the results of the drug metabolism test component 10.

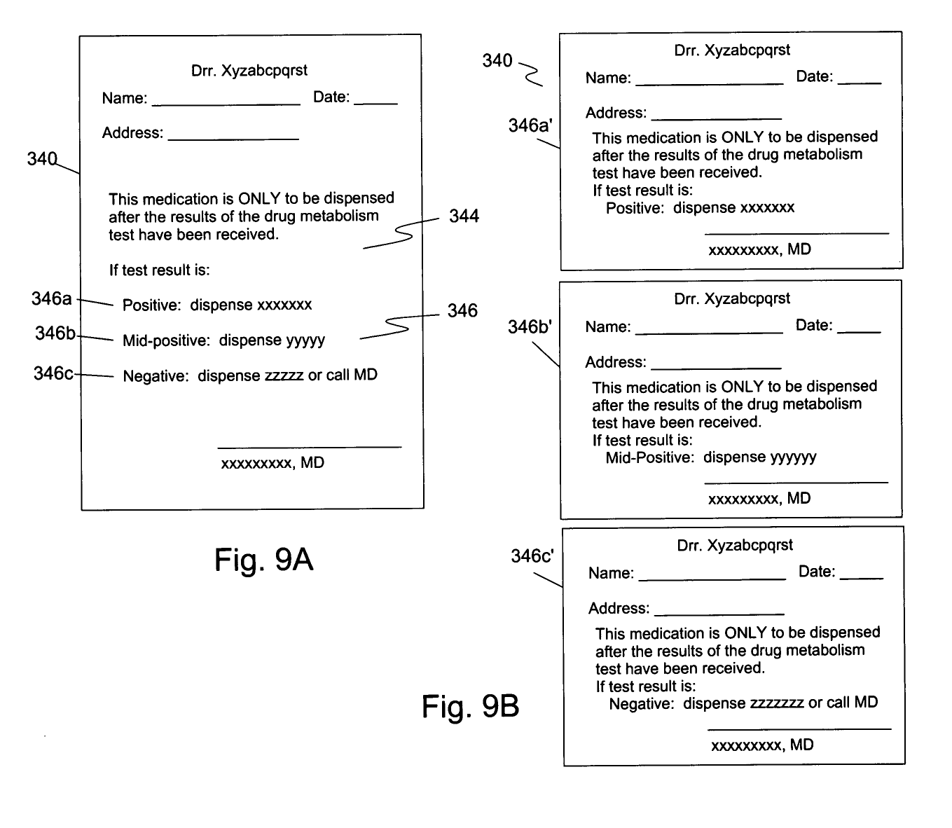

A more efficient way to carry out the intent of the present invention is illustrated in FIGS. 9A and 9B. FIG. 9A shows a prescription component 340 that includes a prescription portion 344. Prescription portion 344 includes multiple, dosing parameter instructions 346 to the pharmacist based on the results of the drug metabolism test component 10. The multiple dosing parameter instructions 346 includes a homozygous positive instruction 346a that indicates dispensing of the prescribed dose, a heterozygous mid-positive instruction 346b that indicates dispensing of an adjusted dose, and a negative instruction 346c that indicates dispensing of a further adjusted dose or that a new medication should be dispensed.

Turning now to FIG. 9B, there is shown variation of the prescription component 340 shown in FIG. 9A. Prescription component 340 includes a first conditional prescription 346a′, a second conditional prescription 346b′, and a third conditional prescription 346c′. First conditional prescription 346a′ is equivalent to homozygous positive instruction 346a and indicates dispensing of the prescribed dose only if a positive result is obtained from the drug metabolism test. Second conditional prescription 346b′ is equivalent to heterozygous mid-positive instruction 346b and indicates dispensing of an adjusted dose only if a mid-positive result is obtained. Third conditional prescription 346c′ is equivalent to negative instruction 346c and indicates dispensing of a further adjusted dose or that a new medication should be dispensed only if a negative result is obtained.

In certain healthcare situations, it is preferable that the patient at least receive an initial dose of medication to begin treatment pending the results of the drug metabolism test component 10. Typically, an initial dose in these situations will not produce an adverse drug event until additional doses are taken. It is the additional doses that increase the level of medication in a patient whose system is unable to properly metabolize the medication at the level taken that leads to the adverse drug event.

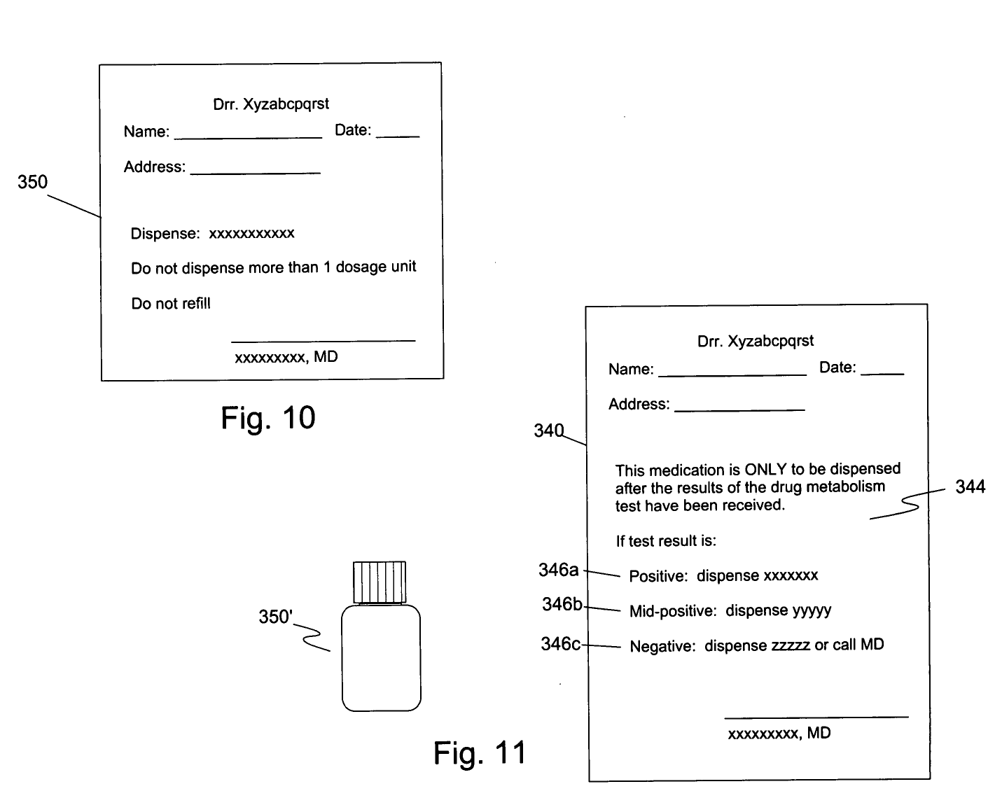

FIG. 10 illustrates another embodiment of the prescription component 340. Prescription component 340 includes an initial dose prescription 350 along with the conditional prescription. Initial dose prescription 350 authorizes the pharmacist to dispense an initial dose of the medical therapy medication but not to dispense or fill any additional dosages until the results of the drug metabolism test component 10 are received. It is noted that, in addition to being a written prescription, initial dose prescription 350 may also be a prescription that has been communicated by telephone, facsimile, computer such as for example by email, or any other means used to communicate the prescription. An alternative to providing initial dose prescription 350, the healthcare provider may dispense the initial dose 350′ of medication directly to the patient along with prescription component 340 that includes a prescription portion 344 of prescription component 340 that includes a first conditional prescription 346a′, a second conditional prescription 346b′, and a third conditional prescription 346c′, as illustrated in FIG. 11.

As contemplated by the present invention, the directed medication system 1 may be in a kit form provided by healthcare provider or by prescription. In the alternative, the directed medication system I may be a combination of an over-the-counter test component 10 linked or coupled to prescription instruction component 300.

To use the preferred embodiment of the directed medication system 1, the user/patient obtains a drug metabolism test component 10 that is preferably a genomics test kit either directly from a healthcare provider such as a medical doctor or by prescription from a healthcare provider such as a pharmacist or from the pharmacy as an over-the-counter item. The patient also obtains an initial dose 350′ of medical therapy and a conditional prescription 340 from the patient's doctor, which is to be filled based on the results of the test component 10.

The user opens test component 10 and removes swabs 30 that have indicating indicia 32 on each side. In this case, indicating indicia 32 are “Right Cheek #1,” “Right Cheek #2,” “Left Cheek #1,” and “Left Cheek #2.” The user takes swab 30 marked “Right Cheek #1,” places the user's thumb over the words that say “Right Cheek #1,” and puts the swab into the user's mouth placing the flat surface against the inside of the right cheek. The user then gently rubs the tip of the swab up and down five times with a motion of about one-half of an inch. This motion results in a few cells lining the inside of the right cheek to stick to swab 30.

The user removes swab 30 from the mouth and swirls the tip of swab 30 containing the cells about ten times onto the second holding pad 24 marked “Right Cheek.” Using the same swab 30, the user is instructed to repeat the process by now placing the user's thumb over the words that say “Right Cheek #2.” After rubbing the tip of swab 30 against the inside of the right cheek, the user swirls the tip of swab 30 containing more cells about ten times onto the first holding pad 24. This process is repeated for the left cheek using swab 30 marked “Left Cheek #1” and “Left Cheek #2.”

Upon completion of sample collection, the sample container 20 is sealed by folding upper portion 26a over lower portion 26b and securing upper portion 26a to lower portion 26b using securing tab 27. The user then removes the peel off tracking number and retains it for reference purposes, and mails the sample container 20 to the indicated address for analysis. It is understood that if the results are to be communicated directly to the patient's pharmacy or healthcare provider, then the appropriate forwarding information will need to be included when submitting the test sample for analysis.

In the preferred embodiment, the results of the test, which is also known as the genomics analysis or the genomics information, are then communicated to either the patient or the patient's healthcare provider who then communicate the test results with other healthcare providers as necessary to customize the medical therapy based on the test results. The customized medical therapy is only dispensed after the test results have been communicated so that the healthcare provider can dispense the appropriate medical therapy based on the test results. The medical therapy is customized according to the test results of the genomics analysis. If the genomics analysis indicates a homozygous positive pattern the prescribed dose is dispensed. If the genomics analysis indicates a heterozygous mid-positive pattern an adjusted dose is dispensed. If the genomics analysis indicates a negative pattern a further adjusted dose is dispensed or a new medication is dispensed.

Although the preferred embodiments of the present invention have been described herein, the above description is merely illustrative. Further modification of the invention herein disclosed will occur to those skilled in the respective arts and all such modifications are deemed to be within the scope of the invention as defined by the appended claims.

What is claimed is:

1. A directed medication system comprising:
a drug metabolism test component comprising a medical sample receiving apparatus configured to receive a user's biological sample for determining the presence of one or more predefined drug metabolism markers; and
a prescription instruction component containing at least coordinating instructions disclosing to a user of said drug metabolism test component the procedure for obtaining a customized medical therapy based on the results of said drug metabolism test component.

2. The system of claim 1 wherein said one or more predefined drug metabolism markers is selected from the group consisting of DNA and enzymes.

3. The system of claim 1 wherein said drug metabolism test component is a genomics-based test.

4. The system of claim 3 wherein said drug metabolism test component comprising:
a hinged folder having at least one absorbent sample holding pad impregnated with chemicals to lyse cell membranes and immobilize nucleic acids wherein said at least one holding pad has a pad indicia, said pad configured for receiving a patient's buccal cell sample;
a buccal cell sampling swab wherein said swab has indicia linking said swab to said indicia; and
test component instructions for collecting, applying and submitting said patient's buccal cell sample for customizing a medical therapy using genomic information derived from said buccal sample of said patient.

5. The system of claim 1 wherein said prescription instruction component includes a first instruction directing said user to obtain said drug metabolism test component and to follow the instructions provided with said drug metabolism test component, and a second instruction directing said user to obtain said customized medical therapy based on said results of said drug metabolism test component.

6. The system of claim 1 wherein said prescription instruction component includes an instruction component and a prescription component wherein said prescription component includes at least a conditional dosage prescription for said customized medical therapy based on said results of said drug metabolism test component.

7. The system of claim 6 wherein said conditional dosage prescription includes a prescription portion instructing a prescription provider on dispensing said customized medical therapy based on the results of said drug metabolism test component.

8. The system of claim 7 wherein said prescription portion includes a homozygous positive test instruction, a heterozygous mid-positive test instruction and a negative test instruction.

9. The system of claim 7 wherein said conditional dosage prescription includes a plurality of separate prescriptions wherein said plurality of separate prescriptions includes a first prescription for a homozygous positive test result, a second prescription for a heterozygous mid-positive test result and a third prescription for a negative test result.

10. The system of claim 6 wherein said prescription component further includes an initial dose prescription.

11. The system of claim 10 wherein said conditional dosage prescription includes a prescription portion instructing a prescription provider on dispensing said customized medical therapy based on the results of said drug metabolism test component.

12. The system of claim 11 wherein said prescription portion includes a homozygous positive test instruction, a heterozygous mid-positive test instruction and a negative test instruction.

13. The system of claim 11 wherein said conditional dosage prescription includes a plurality of separate prescriptions wherein said plurality of separate prescriptions includes a first prescription for a homozygous positive test result, a second prescription for a heterozygous mid-positive test result and a third prescription for a negative test result.

14. The system of claim 10 wherein said initial dose prescription for an initial dose of medical therapy is selected from the group consisting of mercaptopurine, flourouracil, or azathioprene.

15. The system of claim 6 wherein said prescription component further includes an initial dosage of said medical therapy.

16. The system of claim 15 wherein said conditional dosage prescription includes a prescription portion instructing a prescription provider on dispensing said customized medical therapy based on the results of said drug metabolism test component.

17. The system of claim 16 wherein said prescription portion includes a homozygous positive test instruction, a heterozygous mid-positive test instruction and a negative test instruction.

18. The system of claim 16 wherein said conditional dosage prescription includes a plurality of separate prescriptions wherein said plurality of separate prescriptions includes a first prescription for a homozygous positive test result, a second prescription for a heterozygous mid-positive test result and a third prescription for a negative test result.

19. The system of claim 15 wherein said initial dose of medical therapy is a dose of mercaptopurine, flourouracil, or azathioprene.

20. A directed medication system comprising:
a drug metabolism test means for determining the presence of one or more predefined drug metabolism markers; and
a prescription instruction means for obtaining a customized medical therapy based on the results of said drug metabolism test means.

21. The system of claim 20 wherein said drug metabolism test means is selected from the group consisting of DNA and enzymes.

22. The system of claim 20 wherein said drug metabolism test means is a genomics-based test.

23. The system of claim 20 wherein said prescription instruction means includes a first instruction means directing said user to obtain said drug metabolism test means and to follow the instructions provided with said drug metabolism test means, and a second instruction means directing said user to obtain said customized medical therapy based on said results of said drug metabolism test means.

24. The system of claim 20 wherein said prescription instruction means includes an instruction means and a prescription means wherein said prescription means includes at least a conditional dosage prescription for said customized medical therapy based on said results of said drug metabolism test means.

25. The system of claim 24 wherein said conditional dosage prescription includes a prescription portion instructing a prescription provider on dispensing said customized medical therapy based on the results of said drug metabolism test component.

26. The system of claim 25 wherein said prescription portion includes a homozygous positive test instruction, a heterozygous mid-positive test instruction and a negative test instruction.

27. The system of claim 25 wherein said conditional dosage prescription includes a plurality of separate prescriptions wherein said plurality of separate prescriptions includes a first prescription for a homozygous positive test result, a second prescription for a heterozygous mid-positive test result and a third prescription for a negative test result.

28. The system of claim 24 wherein said prescription means further includes an initial dose prescription.

29. The system of claim 28 wherein said conditional dosage prescription includes a prescription portion instructing a prescription provider on dispensing said customized medical therapy based on the results of said drug metabolism test component.

30. The system of claim 29 wherein said prescription portion includes a homozygous positive test instruction, a heterozygous mid-positive test instruction and a negative test instruction.

31. The system of claim 28 wherein said conditional dosage prescription includes a plurality of separate prescriptions wherein said plurality of separate prescriptions includes a first prescription for a homozygous positive test result, a second prescription for a heterozygous mid-positive test result and a third prescription for a negative test result.

32. The system of claim 28 wherein said initial dose prescription is for an initial dose of medical therapy selected from the group consisting of mercaptopurine, flourouracil, or azathioprene.

33. The system of claim 24 wherein said prescription means further includes an initial dosage of said medical therapy.

34. The system of claim 33 wherein said conditional dosage prescription includes a prescription portion instructing a prescription provider on dispensing said customized medical therapy based on the results of said drug metabolism test component.

35. The system of claim 34 wherein said prescription portion includes a homozygous positive test instruction, a heterozygous mid-positive test instruction and a negative test instruction.

36. The system of claim 34 wherein said conditional dosage prescription includes a plurality of separate prescriptions wherein said plurality of separate prescriptions includes a first prescription for a homozygous positive test result, a second prescription for a heterozygous mid-positive test result and a third prescription for a negative test result.

37. The system of claim 33 wherein said initial dose of medical therapy is a dose of mercaptopurine, flourouracil, or azathioprene.

38. A method for customizing a medical therapy for a patient, said method comprising:
obtaining the results of a drug metabolism test for said patient; and
prescribing a customized medical therapy based on said results.

39. The method of claim 38 further comprising providing said drug metabolism test to said patient.

40. The method of claim 38 further comprising instructing said patient to obtain a drug metabolism test and to follow the instructions of said drug metabolism test.

41. The method of claim 38 further comprising prescribing an initial dose of said medical therapy.

42. The method of claim 38 further comprising providing an initial dose of said medical therapy.

43. A method of a treating medical disorder, said method comprising:
screening a patient for potential adverse reactions to a medical therapy; and
instructing said patient on the mechanism to obtain a customized medical therapy based on the results of said screening step.

44. The method of claim 43 wherein said screening step further includes instructing said patient to obtain a drug metabolism test kit and to follow the instructions of said drug metabolism test kit.

45. The method of claim 43 wherein said instructing step further includes providing a prescription for a customized medical therapy based on the results of a drug metabolism test for said patient.

46. The method of claim 45 wherein said instructing step further includes providing a conditional prescription for a customized medical therapy having a prescription portion that includes a homozygous positive test instruction, a heterozygous mid-positive test instruction and a negative test instruction wherein only one of said test instructions are followed based on the results of said drug metabolism test.

47. The method of claim 45 wherein said instructing step further includes providing a plurality of prescriptions wherein said plurality of prescriptions includes a first conditional prescription for a homozygous positive test result, a second conditional prescription for a heterozygous mid-positive test result and a third conditional prescription for based on the results of said drug metabolism test.

48. The method of claim 45 wherein said prescription providing step further includes providing a prescription for an initial dose of said medical therapy.

49. The method of claim 48 wherein said instructing step further includes providing a conditional prescription for a customized medical therapy having a prescription portion that includes a homozygous positive test instruction, a heterozygous mid-positive test instruction and a negative test instruction wherein only one of said test instructions are followed based on the results of said drug metabolism test.

50. The method of claim 48 wherein said instructing step further includes providing a plurality of prescriptions wherein said plurality of prescriptions includes a first conditional prescription for a homozygous positive test result, a second conditional prescription for a heterozygous mid-positive test result and a third conditional prescription for a negative test result wherein said plurality of prescriptions are based on the results of said drug metabolism test.

51. The method of claim 45 wherein said prescription providing step further includes providing an initial dose of said medical therapy.

52. The method of claim 51 wherein said instructing step further includes providing a conditional prescription for a customized medical therapy having a prescription portion that includes a homozygous positive test instruction, a heterozygous mid-positive test instruction and a negative test instruction wherein only one of said test instructions are followed based on the results of said drug metabolism test.

53. The method of claim 51 wherein said instructing step further includes providing a plurality of prescriptions wherein said plurality of prescriptions includes a first conditional prescription for a homozygous positive test result, a second conditional prescription for a heterozygous mid-positive test result and a third conditional prescription for a negative test result wherein said plurality of prescriptions are based on the results of said drug metabolism test.
